(19) United States
(12) Patent Application Publication
(10) Pub. No.: US 2009/0012321 A1
(43) Pub. Date: Jan. 8, 2009

(54) PROCESS FOR PREPARING 17ALPHA-ACETOXY-6-METHYLENEPREGN-4-ENE-3,20-DIONE, MEDROXYPROGESTERONE ACETATE AND MEGESTROL ACETATE

(76) Inventors: Klaus ANNEN, Munster-Roxel (DE); Thomas Linz, Berlin (DE); Karl-Heinz Neff, Frondenberg (DE); Rolf Bohlmann, Berlin (DE); Henry Laurent, Berlin (DE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 12/133,638

(22) Filed: Jun. 5, 2008

Related U.S. Application Data
(60) Provisional application No. 60/942,285, filed on Jun. 6, 2007.

(51) Int. Cl. C07J 7/00

(52) U.S. Cl. 552/598

(57) ABSTRACT

The present invention relates to a process for preparing 17α-acetoxy-6-methylenepregn-4-ene-3,20-dione (4) as an intermediate, and to processes for preparing medroxyprogesterone acetate (1) (17α-acetoxy-6α-methylpregn-4-ene-3,20-dione) and megestrol acetate (2) (17α-acetoxy-6-methylpregna-4,6-diene-3,20-dione) via this intermediate (4).

This application claims the benefit of the filing date of U.S. Provisional Application Ser. No. 60/942,285, filed Jun. 6, 2007.

The present invention relates to a process for preparing 17α-acetoxy-6-methylenepregn-4-ene-3,20-dione (4) as an intermediate, and to processes for preparing medroxyprogesterone acetate (1) (17α-acetoxy-6α-methylpregn-4-ene-3,20-dione) and megestrol acetate (2) (17α-acetoxy-6-methylpregna-4,6-diene-3,20-dione) via this intermediate (4).

DE 3004508 describes the direct 6-methylenation of 3-oxo-Δ⁴ steroids by means of a formaldehyde acetal in the presence of phosphorus oxychloride. EP 0326340, the closest prior art, describes the disadvantages of the direct of 6-methylenation and the advantages of the Mannich reaction to the 6-methylenation of androsta-4-ene-3,17-diones.

The subject-matter of the present invention differs from that of EP 0326340 in that the steroid to be 6-methylenated is 17α-acetoxypregn-4-ene-3,20-dione (by the 17-substituent).

It is therefore an object of the present invention to provide an alternative process for preparing 17α-acetoxy-6-methylenepregn-4-ene-3,20-dione (4) as an intermediate, and processes for preparing medroxyprogesterone acetate (1) (17α-acetoxy-6α-methylpregn-4-ene-3,20-dione) and megestrol acetate (2) (17α-acetoxy-6-methylpregna-4,6-diene-3,20-dione) via this intermediate (4).

The object is achieved by a process for preparing the 17α-acetoxy-6-methylenepregn-4-ene-3,20-dione intermediate (4)

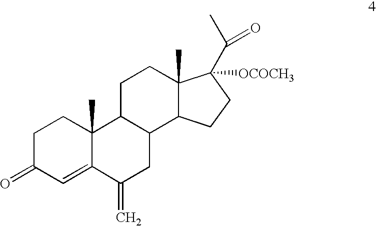

comprising the steps of
a) the conversion of 17α-acetoxypregn-4-ene-3,20-dione (3)

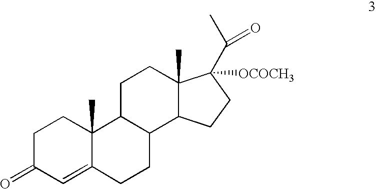

in a Mannich reaction, and
b) subsequent Hofmann elimination.

The present invention further relates to a process for preparing 17α-acetoxy-6α-methylpregn-4-ene-3,20-dione (1),

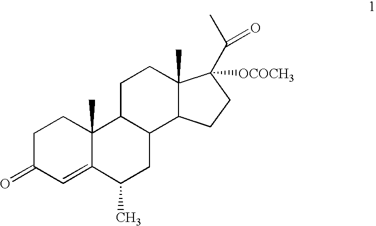

from 17α-acetoxypregn-4-ene-3,20-dione of the formula (3), comprising steps a) and b), wherein, after step b), the compound of the formula (4) is hydrogenated in step c).

The present invention also relates to a process for preparing 17α-acetoxy-6-methyl-pregna-4,6-diene-3,20-dione (2)

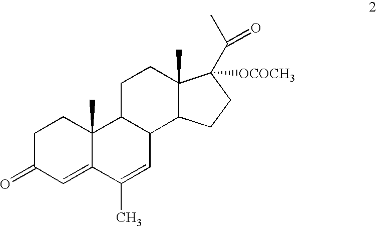

comprising steps a) and b), wherein, after step b), the compound of the formula (4) is isomerized in step d).

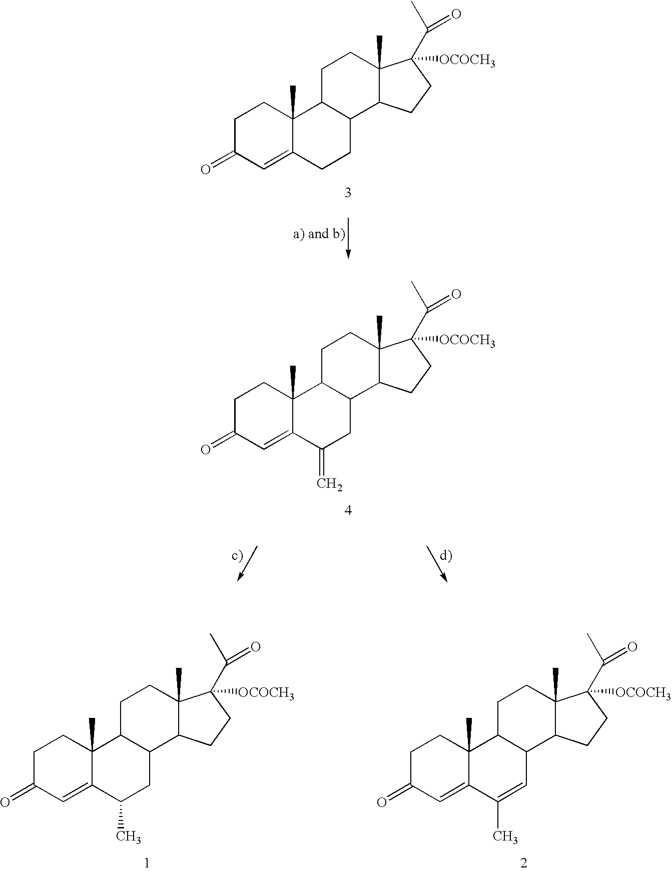

Preference is given according to the present invention to dissolving or suspending the reagents in the Mannich reaction in ethylene glycol dimethyl ether or pure tetrahydrofuran. The solvents described in EP 0326340, for example dioxane, methanol, ethanol, are incapable of suppressing side reactions such as the bis-Mannich reaction ((3) is 2,6-dimethylenated) and the retro-Mannich reaction (the starting compound (3) is reformed). The by-products formed can be separated from the compounds (1) or (2) only with exceptional difficulty or only by complicated column chromatography.

The preferred procedure variant also has the advantage that it enables isolation and hence specific prepurification of the 6-methylamino compound formed in step a) as a precursor of the Hofmann elimination (in step b), which would otherwise not be possible with the solvents described in EP 0326340.

In the process according to the invention, the intermediate (4) without chromatographic purification already has a purity of at least 97.5%, in contrast to the qualities achievable in the known processes (DE 3004508, EP 0 326 340 A2) (purity below 95.5%). The higher purity of the intermediate (4) additionally has the effect that it can be fully hydrogenated to the compound (1) or isomerized to the compound (2) with significantly smaller amounts of catalyst, and that 17α-acetoxy-6α-methylpregn-4-ene-3,20-dione (1) and 17α-acetoxy-6-methylpregna-4,6-diene-3,20-dione (2) can be obtained in very high purity.

To perform the Mannich reaction (step a), it is typically possible to employ primary or secondary amines (Synthesis 1973, 703-775). Suitable amines are, for example, those of the formula (5),

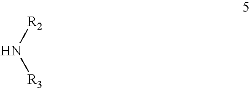

in which R₂ and R₃ together are the —(CH₂)₂-Q-(CH₂)₂— moiety where Q is defined as a carbon-carbon bond, a methylene group or an oxygen atom, or in which R₂ is an alkyl group having up to eight carbon atoms, a benzyl group or a phenyl radical optionally substituted by alkyl groups or alkoxy groups containing up to 4 carbon atoms and/or fluorine atoms or chlorine atoms, and R₃ is as defined for R₂ or represents a hydrogen atom.

In the Mannich reactions performed to date, the N-methylaniline was used as the amine component. However, other amines are also suitable, for example pyrrolidine, piperidine, morpholine, diethylamine, diisopropylamine or N-methylbenzylamine.

The Hofmann elimination (step b) is preferably performed in the presence of acids as catalysts (for example mineral acids, such as hydrogen chloride, sulphuric acid or phosphoric acid, or preferably strong organic acids such as trifluoroacetic acid, methanesulphonic acid or trifluoromethanesulphonic acid). In step b), preference is given to subjecting the isolated and optionally purified intermediate from step a) to the Hofmann elimination dissolved or suspended in ethylene glycol dimethyl ether or pure tetrahydrofuran.

The purity of the 6-methylene compound of the formula (4) is sufficiently high that, for the subsequent hydrogenation to the compound of the formula (1) or for the isomerization to the compound of the formula (2), compared to the Vilsmeier process, only very small amounts of catalyst (Pd/C) are required.

The intermediate of the formula (4) is hydrogenated to the compound of the formula (1) by hydrogen transfer from cyclohexene in the presence of a palladium/carbon catalyst (Chem. Soc., London; 1954, 3578f.). To perform the hydrogenation, the components are dissolved or suspended in a polar solvent, preferably ethanol. After the workup, 17α-acetoxy-6α-methylpregn-4-ene-3,20-dione of the formula (1) is obtained in good yield and in outstanding purity (reference is made to the content determination in Example 1 adduced below).

The intermediate of the formula (4) is isomerized in a similar manner to the hydrogenation, but in the presence of a catalytic amount of 4-methyl-1-cyclohexene instead of cyclohexene. After the workup, 17α-acetoxy-6-methylpregna-4,6-diene-3,20-dione of the formula (2) is obtained in good yield, and likewise satisfies very high purity requirements (reference is made to the content determination in Example 2 adduced below).

The process according to the invention allows medroxyprogesterone acetate (1) and megestrol acetate (2) to be prepared in a purity of ≧99.0%.

The working examples which follow serve to illustrate the process according to the invention.

### EXPERIMENTAL SECTION

### Example 1

### Process for Preparing Medroxyprogesterone Acetate (1)

i.) A suspension of 17α-acetoxypregn-4-ene-3,20-dione in 2 parts of ethylene glycol dimethyl ether and 0.6 part of triethyl orthoformate is stirred while heating, admixed with 0.05 part of methanesulphonic acid and stirred until complete conversion. After 0.3 part of N-methylaniline and 4 parts of ethylene glycol dimethyl ether have been added, 0.23 part of formaldehyde (about 37% strength) is metered in within 30 minutes. The reaction mixture is then stirred at at least 40° C. On completion of conversion, the mixture is cooled, 4.8 parts of water are metered in and the mixture is stirred until complete precipitation. The moist substance removed is extracted by stirring in 2.8 parts of diisopropyl ether, isolated and washed with diisopropyl ether. The 6-methylamino compound obtained is suspended in 4 parts of ethylene glycol dimethyl ether, and admixed under cold conditions with one part of conc. hydrochloric acid. On completion of conversion, at room temperature, the solid substance is removed, and washed with ethylene glycol dimethyl ether and then with methanol. The moist substance is suspended in 1.4 parts of dimethylformamide under hot conditions. At room temperature, the crystals are centrifuged off and washed with dimethylformamide and then with water. After drying, 0.83 part of 17α-acetoxy-6-methylenepregn-4-ene-3,20-dione of melting point 239-240° C. are obtained; [α]_{D}²⁰=+260° (chloroform). Content: 99.3% (HPLC).
ii.) A suspension of 0.03 part of palladium/carbon (type E101 N/W) and 0.02 part of sodium acetate in a mixture of 5 parts of ethanol, 0.2 part of water and 2 parts of cyclohexene is heated to boiling. A suspension of 1 part of 17α-acetoxy-6-methylenepregn-4-ene-3,20-dione in 5 parts of ethanol and 0.16 part of water is prepared and this suspension is added to the catalyst suspension. The end of the hydrogenation is determined by gas chromatography. On completion of conversion, the mixture is cooled, 3 parts of methylene chloride are added and the mixture is filtered through a heated pressure filter. The filter residue is washed with warm methylene chloride and with warm ethanol. The wash solutions are added to the filtrate. The filtrate is adjusted to a pH of 1 by adding conc. hydrochloric acid and concentrated under reduced pressure to a residual amount of bottoms of 3.5 parts. For complete precipitation, 5 parts of water are added and the mixture is stirred. At room temperature, the crystals are removed and washed with water. The moist product is suspended in a mixture of 5 parts of ethanol/water (1/1) and 0.01 part of conc. hydrochloric acid under hot conditions and removed after cooling. The crystals are washed with a mixture of ethanol/water (1/1;V/V) and then with water. 0.82 part of medroxyprogesterone acetate is isolated, melting point 206-208° C.; [α]_{D}²⁰=+48° (chloroform). Content: 100.3% (HPLC).

### Example 2

### Process for Preparing Megestrol Acetate (2)

i.) 17α-Acetoxy-6-methylenepregn-4-ene-3,20-dione (4) is prepared in the process as described in Example 1 (i.).
ii.) A suspension of one part 17α-acetoxy-6-methylenepregn-4-ene-3,20-dione, 0.1 part of sodium acetate and 0.06 part of palladium/carbon in 10 parts of ethanol and 0.01 part of 4-methyl-1-cyclohexene is heated at reflux with stirring, and the completeness of isomerization is determined by thin-layer chromatography. 1.5 parts of ethanol are added and the catalyst is filtered off under hot conditions, and is washed with ethanol. Combined wash liquors and filtrate are concentrated to 3 parts. For complete precipitation, 7.5 parts of water are added, and the crystals are removed and washed with water. After drying, 0.98 part of crude megestrol acetate is obtained, which is dissolved under hot conditions in 12 parts of methanol. The mixture is concentrated to a residual amount of bottoms of 2.4 parts, cooled and centrifuged. The moist substance is dissolved under hot conditions in 12 parts of methanol and, after concentration to a residual volume of 2 parts, cooled. The solid substance is removed, washed with methanol, and dissolved in 8 parts of acetone. After 0.05 part of activated carbon has been added, the mixture is heated to boiling, the activated carbon is filtered off and the filtrate is concentrated to a residual amount of bottoms of 1 part. The suspension is cooled, and the crystals are removed and washed with acetone. After drying, 0.85 part of megestrol acetate with a melting point of 217-220° C. and [α]_{D}²⁵=+11° (chloroform) is isolated. Content: 100.3% (HPLC).

Without further elaboration, it is believed that one skilled in the art can, using the preceding description, utilize the present invention to its fullest extent. The preceding preferred specific embodiments are, therefore, to be construed as merely illustrative, and not limitative of the remainder of the disclosure in any way whatsoever.

In the foregoing and in the examples, all temperatures are set forth uncorrected in degrees Celsius and, all parts and percentages are by weight, unless otherwise indicated.

The entire disclosures of all applications, patents and publications, cited herein and of corresponding U.S. Provisional Application Ser. No. 60/942,285, filed Jun. 6, 2007, are incorporated by reference herein.

The preceding examples can be repeated with similar success by substituting the generically or specifically described reactants and/or operating conditions of this invention for those used in the preceding examples.

From the foregoing description, one skilled in the art can easily ascertain the essential characteristics of this invention and, without departing from the spirit and scope thereof, can make various changes and modifications of the invention to adapt it to various usages and conditions.

1. Process for preparing 17α-acetoxy-6-methylenepregn-4-ene-3,20-dione (4) 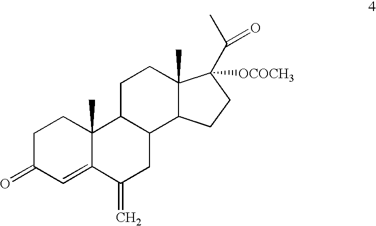
comprising the steps of
a) the conversion of 17α-acetoxypregn-4-ene-3,20-dione (3) 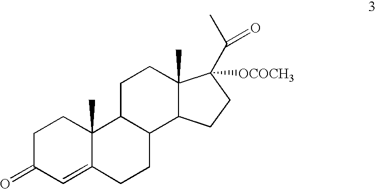
in a Mannich reaction, and
b) subsequent Hofmann elimination.

2. Process for preparing 17α-acetoxy-6α-methylpregn-4-ene-3,20-dione (1), 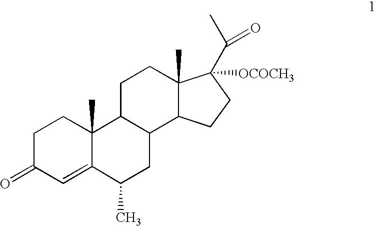
according to claim 1, wherein, after step b),
the compound (4) is hydrogenated in step c).

3. Process for preparing 17α-acetoxy-6-methylpregna-4,6-diene-3,20-dione (2) 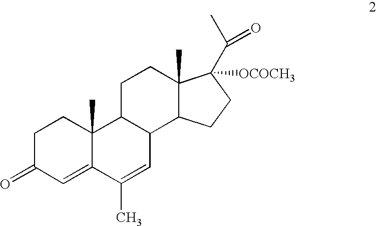
according to claim 1, wherein, after step b),
the compound (4) is isomerized in step d).

4. Process according to claim 1, characterized in that, in step a), the reagents are dissolved or suspended in ethylene glycol dimethyl ether or pure tetrahydrofuran.

5. Process according to claim 1, characterized in that the Mannich reaction in step a) is performed with N-methylaniline, a formaldehyde solution, triethyl orthoformate and methanesulphonic acid.

6. Process according to claim 1, characterized in that, in step a), the intermediate is isolated and purified.

7. Process according to claim 1, characterized in that, in step b), ethylene glycol dimethyl ether or pure tetrahydrofuran is used as a solvent.
